(19) United States
(12) Patent Application Publication
(10) Pub. No.: US 2021/0228770 A1
(43) Pub. Date: Jul. 29, 2021

(54) A SYNTHETIC OPHTHALMIC GRAFT PATCH

(71) Applicant: CORNEAT VISION LTD., Ra'anana (IL)

(72) Inventor: Gilad LITVIN, MoshavSde Varburg (IL)

(73) Assignee: CORNEAT VISION LTD., Ra'anana (IL)

(21) Appl. No.: 15/734,607

(22) PCT Filed: Jun. 5, 2019
(86) PCT No.: PCT/IL2019/050640

§ 371 Date: Dec. 3, 2020

Related U.S. Application Data
(60) Provisional application No. 62/681,082, filed on Jun. 5, 2018.

(51) Int. Cl. A61L 27/14; A61L 27/54; A61L 27/56; A61L 27/58

(52) U.S. Cl. CPC A61L 27/14; A61L 27/54; A61L 2430/16; A61L 27/58; A61L 2400/12; A61L 27/56

(57) ABSTRACT

The present invention provides synthetic ophthalmic graft patches, including devices comprising them, and uses thereof in ophthalmic tissue replacement therapies and ophthalmic tissue reconstruction/regeneration therapies.

### BACKGROUND OF THE INVENTION

It is estimated that 285 million people worldwide are visually impaired, of whom 39 million are blind. Corneal opacities and trachoma alone are estimated to account for 4% and 3% of world blindness, respectively, ranking corneal blindness behind only cataract (51%) and glaucoma (8%). Nearly 185,000 corneal transplants are performed each year in over 115 different countries, with nearly 80,000 performed in the US alone. Of the corneal grafts used worldwide, 87% are procured from donors within the same country, while 27 countries (1.2% of corneal transplants) rely solely on imported corneas to supply their need for corneal allografts. Limited access to viable graft tissue remains a challenge in many parts of the world, leaving over half of the world's population without access to corneal transplantation services.

Scleral thinning is a well-reported complication following pterygium excision, glaucoma related surgery, retinal detachment repair, systemic diseases such as vasculitis, high myopia, or trauma. In some cases, it results in staphyloma formation, scleral perforation, and uveal exposure. Reinforcement of thin or perforated sclera is necessary, especially when the choroid is exposed to prevent prolapse of ocular contents and secondary infection. Various types of grafts have been used in this situation, but none has been uniformly accepted. Scleral grafts are typically available from donor eyes. Failure of scleral grafts has been reported owing to lack of vascularization with resultant necrosis, sloughing and/or gradual degradation.

Eye banks are institutions responsible for collecting, processing, and distributing donated ocular tissue for transplantation, helping to mitigate this disparity between harvested ocular tissue supply and demand.

Since the grafts are derived from donors there are different potential adverse events associated with corneal allograft transplantation including: infectious disease and serology (such as HIV), viral hepatitis, syphilis, endophthalmitis, sepsis, noninfectious systemic disease transmission, malignancy, prion disease and so forth.

Due to infectious and communicable diseases, increased regulation, eye banks cannot provide the increasing need and challenge of safe, high-quality, and timely tissue for any type of ophthalmic transplantation.

### SUMMARY OF THE INVENTION

The present invention provides a synthetic ophthalmic graft patch having a porous polymeric structure with pores of less than 5 microns. The invention further provides a synthetic ophthalmic graft patch having a porous polymeric structure with pores of between 5 and 20 micros.

When referring to a “synthetic ophthalmic graft patch”, it should be understood to encompass any type of synthetic artificial tissue substitute designated to be used to replace or complement any part of the eyeball and/or orbital anatomy. For example, said synthetic graft patch of the invention, may be used in ophthalmic implantation or transplantation procedures. In some examples said synthetic graft patch of the invention may be used to replace a diseased tissue of any part of the eyeball and/or orbit of a subject in need thereof. In other examples said synthetic graft patch of the invention may be used to complement or be added to an implantable device used in an ophthalmic procedure.

It is to be understood that a synthetic ophthalmic graft patch of the invention can be in any shape or form suitable for the procedure to be performed and for the part of the anatomical eye part that is being treated. In some embodiments, the shape of a synthetic ophthalmic graft patch of the invention is concaved. In other embodiments, the shape of a synthetic ophthalmic graft patch of the invention is convexed. In some embodiments, the shape of a synthetic ophthalmic graft patch of the invention is in the form of a tube. In some embodiments, the shape of a synthetic ophthalmic graft patch of the invention is in the form of at least part of the sclera of a patient. In some embodiments, the shape of a synthetic ophthalmic graft patch of the invention is in the form of at least part of the conjunctiva of a patient. In some embodiments, the shape of a synthetic ophthalmic graft patch of the invention is in the form of at least part of the cornea of a patient. In some embodiments, the shape of a synthetic ophthalmic graft patch of the invention is in the form of at least a part of the eyelid, optionally with the tarsus, of a patient. In some embodiments, the shape of a synthetic ophthalmic graft patch of the invention is in the form of at least a part of the lacrimal tube of a patient. In some embodiments, the shape of a synthetic ophthalmic graft patch of the invention is in the form of at least a part of the tenon of a patient.

Said synthetic graft patch of the invention is defined to have a porous polymeric structure with pores of less than 5 microns. In other embodiments said pores have a size of between 0.1 to 5 microns. In other embodiments said pores have a size of between 0.1 to 4 microns. In other embodiments said pores have a size of between 0.1 to 3 microns. In other embodiments said pores have a size of between 0.1 to 2 microns. In other embodiments said pores have a size of between 0.1 to 1 microns. In other embodiments, said pored have a size of 0.1, 0.2, 0.3, 0.5\4, 0.5, 0.6, 0.7, 0.8, 0.9, 1.0, 1.5, 2.0, 2.5, 3.0, 3.5, 4.0, 4.5 or 5 microns.

In some embodiments, said pores have a size of between 5 to 20 microns. In some embodiments, said pores have a size of between 5 to 10 microns. In some embodiments, said pores have a size of between 5 to 15 microns. In some embodiments, said pores have a size of between 5 to 7 microns. In some embodiments, said pores have a size of 5, 6, 7, 8, 9, 10, 11, 12, 13, 14, 15, 16, 17, 18, 19 or 20 microns.

In some embodiments, said synthetic ophthalmic graft patch of the invention is a monolayered patch (i.e. it is constructed of a single layer of said porous polymeric structure). In other embodiments, said synthetic ophthalmic graft patch of the invention is a multi-layered patch (i.e. it is constructed of at least two layers of said porous polymeric structure, which may be the same or different).

In some embodiments, said synthetic ophthalmic graft patch of the invention is a biocompatible patch (i.e. the graft patch of the invention is suitable to maintain long and/or short-term functionality compatible with the ophthalmic tissues it is replacing or complementing).

In other embodiments, said synthetic ophthalmic graft patch of the invention is a biodegradable patch (i.e. said graft patch of the invention disintegrates after a predetermined time period).

In some embodiments, said synthetic ophthalmic graft patch of the invention has a thickness of at least 50 microns. In other embodiments, said synthetic ophthalmic graft patch of the invention has a thickness of between about 50 to about 250 micrometers. In other embodiments, said graft patch thickness is about 50, 60, 70, 80, 90, 100, 110, 120, 130, 140, 150, 160, 170, 180, 190, 200, 210, 220, 230, 240, 250 microns In other embodiments said ophthalmic graft patch has a thickness of at least 250 microns. In other embodiments, said graft patch thickness is between about 250 to about 2500 microns. In other embodiments, said graft patch thickness is about 250, 300, 350, 400, 450, 500, 550, 600, 650, 700, 750, 800, 850, 900, 950, 1000, 1100, 1200, 1300, 1400, 1500, 1600, 1700, 1800, 1900, 2000, 2100, 2200, 2300, 2400, 2500 microns.

In other embodiments, said porous polymeric structure comprises at least one polymer. In other embodiments, said porous polymeric structure comprises at least two different polymers (difference may be related to any property including chemical properties (including but not limited to type of compounds, monomers, oligomers, stereochemistry and so forth), physical properties (including but not limited to length, pore size, flexibility, hydrophilicity, magnetic properties), biological properties (including but not limited to biocompatibility, biodegradability and so forth) of the polymers and any combination of properties thereof).

In further embodiments, said porous polymeric structure comprises nanofibers.

In other embodiments, said porous polymeric structure comprises at least one porous electrospun polymer.

In further embodiments, said porous polymeric structure comprises at least one polymer selected from poly(DTE carbonate) polycaprolactone (PCL), polylactic acid (PLA), poly-L-lactic acid (PLLA), Poly(DL-lactide-co-caprolactone, Poly(ethylene-co-vinyl acetate) vinyl acetate, Poly(methyl methacrylate), Poly(propylene carbonate), Poly(vinylidene fluoride), Polyacrylonitrile, Polycaprolactone, Polycarbomethylsilane, Polylactic acid, Polystyrene, Polyvinylpyrrolidone, poly vinyl alcohol (PVA), polyethylene oxide (PEO), polyurethane, polyvinyl chloride (PVC), hyaluronic acid (HA), chitosan, alginate, polyhydroxybuyrate and its copolymers, Nylon 11, Cellulose acetate, hydroxyappetite, poly(3-hydroxybutyric acid-co-3-hydroxyvaleric acid), poly(DL-lactide), polycaprolactone, and poly(L-lactide) or any combination thereof.

Electrospun fibers are typically several orders in magnitude smaller than those produced using conventional spinning techniques. By optimizing parameters such as: i) the intrinsic properties of the solution including the polarity and surface tension of the solvent, the molecular weight and conformation of the polymer chain, and the viscosity, elasticity, and electrical conductivity of the solution; and ii) the operational conditions such as the strength of electric field, the distance between spinneret and collector, and the feeding rate of the solution, electrospinning is capable of generating fibers as thin as tens of nanometers in diameter. Additional parameters that affect the properties of electrospun fiber include the molecular weight, molecular-weight distribution and structure (branched, linear etc.) of the polymer, solution properties (viscosity, conductivity and surface tension), electric potential, flow rate and concentration, distance between the capillary and collection screen, ambient parameters (temperature, humidity and air velocity in the chamber), motion of target screen (collector) and so forth. Fabrication of highly porous fibers may be achieved by electrospinning the jet directly into a cryogenic liquid. Well-defined pores developed on the surface of each fiber as a result of temperature-induced phase separation between the polymer and the solvent and the evaporation of solvent under a freeze-drying condition.

Several approaches have been developed to organize electrospun fibers into aligned arrays. For example, electrospun fibers can be aligned into a uniaxial array by replacing the single-piece collector with a pair of conductive substrates separated by a void gap. In this case, the nanofibers tend to be stretched across the gap oriented perpendicular to the edges of the electrodes. It was also shown that the paired electrodes could be patterned on an insulating substrate such as quartz or polystyrene so the uniaxially aligned fibers could be stacked layer-by-layer into a 3D lattice. By controlling the electrode pattern and/or the sequence for applying high voltage, it is also possible to generate more complex architectures consisting of well-aligned nanofibers.

Electrospun nanofibers could also be directly deposited on various objects to obtain nanofiber-based constructs with well-defined and controllable shapes. In addition, one can manually process membranes of aligned or randomly oriented nanofibers into various types of constructs after electrospinning: for example, fabrication of a tube by rolling up a fibrous membrane or the preparation of discs with controllable diameters by punching a fibrous membrane.

The present invention relates to any eletrospinning technique known in the art, which includes Electrospinning, J. Stanger, N. Tucker, and M. Staiger, I-Smithers Rapra publishing (UK), An Introduction to Electrospinning and Nanofibers, S. Ramakrishna , K. Fujihara, W-E Teo, World Scientific Publishing Co. Pte Ltd (June 2005), Electrospinning of micro- and nanofibers: fundamentals and applications in separation and filtration processes, Y. Fillatov, A. Budyka, and V. Kirichenko (Trans. D. Letterman), Begell House Inc., New York, USA, 2007, which are all incorporated herein by reference in their entirety.

Suitable electrospinning techniques are disclosed, e.g., in International Patent Application, Publication Nos. WO 2002/049535, WO 2002/049536, WO 2002/049536, WO 2002/049678, WO 2002/074189, WO 2002/074190, WO 2002/074191, WO 2005/032400 and WO 2005/065578, the contents of which are hereby incorporated by reference. It is to be understood that although the according to the presently preferred embodiment of the invention is described with a particular emphasis to the electrospinning technique, it is not intended to limit the scope of the invention to the electrospinning technique. Representative examples of other spinning techniques suitable for the present embodiments include, without limitation, a wet spinning technique, a dry spinning technique, a gel spinning technique, a dispersion spinning technique, a reaction spinning technique or a tack spinning technique. Such and other spinning techniques are known in the art and disclosed, e.g., in U.S. Pat. Nos., 3,737,508, 3,950,478, 3,996,321, 4,189,336, 4,402,900, 4,421,707, 4,431,602, 4,557,732, 4,643,657, 4,804,511, 5,002,474, 5,122,329, 5,387,387, 5,667,743, 6,248,273 and 6,252,031 the contents of which are hereby incorporated by reference.

In some embodiments, said synthetic ophthalmic graft patch of the invention further comprises at least one active agent.

In some embodiments, said at least one active agent is selected from a protein, collagen, fibronectin, or TGF-beta 2, heparin, growth factors, antibodies, antimetabolites, chemotherapeutic agents, anti-inflammatory agent, antibiotic agent, antimicrobial agent, and any combinations thereof.

The invention further provides a synthetic ophthalmic graft patch as disclosed herein and above being a tissue replacement patch.

The invention further provides a synthetic ophthalmic graft patch as disclosed herein and above being a tissue supplement patch.

The invention further provides a synthetic ophthalmic graft patch as disclosed herein and above being a tissue reconstruction/regeneration patch.

The invention further provides a synthetic ophthalmic graft patch as disclosed herein being at least a part of at least one of a sclera, a conjunctiva, cornea, an eyelid tarsus, lacrimal tube, a tenon of the eye of a patient, and any combinations thereof.

The invention further provides a synthetic ophthalmic graft patch as disclosed herein for use in ophthalmic tissue replacement procedures. The invention further provides a synthetic ophthalmic graft patch as disclosed herein for use in ophthalmic tissue supplement procedures. The invention further provides a synthetic ophthalmic graft patch as disclosed herein for use in ophthalmic tissue reconstruction/regeneration procedures. The invention further provides a synthetic ophthalmic graft patch as disclosed herein for use in.

The invention provides a synthetic ophthalmic graft patch of the invention for use in ophthalmic tissue replacement therapy. The invention further provides a synthetic ophthalmic graft patch of the invention for use in ophthalmic tissue reconstruction/regeneration therapy.

In some embodiments, said ophthalmic tissue replacement and/or ophthalmic tissue reconstruction and/or ophthalmic tissue regeneration therapies are selected from eyelid tarsus supplement procedures, reinforcement of implants (for example for covering glaucoma tube implants or shunts in order to minimize the potential of tube erosion), correction of hypotony in an over-filtering bleb, scleral reinforcement (for example if there is an area of auto-filtration), repair of an eroded scleral buckle, anterior segment reconstruction, treatment of ocular tumors requiring radiotherapy, scleral reinforcement for scleromalacia, cryotherapy, scleral resection of ocular tumors and any combinations thereof.

The invention further provides a synthetic ophthalmic graft patch as disclosed herein for use in covering ophthalmic implants (for example for covering glaucoma tube implants or shunts in order to minimize the potential of tube erosion).

The invention further provides a synthetic ophthalmic graft patch as disclosed herein for use in correcting hypotony in an over-filtering bleb. The invention further provides a synthetic ophthalmic graft patch as disclosed herein for use in scleral reinforcement (for example if there is an area of auto-filtration). The invention further provides a synthetic ophthalmic graft patch as disclosed herein for use in the repair of an eroded scleral buckle. The invention further provides a synthetic ophthalmic graft patch as disclosed herein for use in anterior segment reconstruction. The invention further provides a synthetic ophthalmic graft patch as disclosed herein for use in conjunction with treatment of ocular tumors requiring radiotherapy. The invention further provides a synthetic ophthalmic graft patch as disclosed herein for use in scleral reinforcement for scleromalacia. The invention further provides a synthetic ophthalmic graft patch as disclosed herein for use in cryotherapy, or scleral resection of ocular tumors.

The invention further provides a device comprising at least one synthetic ophthalmic graft patch as defined herein above and below.

### BRIEF DESCRIPTION OF THE DRAWINGS

The subject matter regarded as the invention is particularly pointed out and distinctly claimed in the concluding portion of the specification. The invention, however, both as to organization and method of operation, together with objects, features, and advantages thereof, may best be understood by reference to the following detailed description when read with the accompanying drawings in which:

FIG. 1A, FIG. 1B and FIG. 1C show a scheme of a synthetic ophthalmic graft patch of the invention wherein its capacity in eyelid tarsus supplement procedures.

FIG. 2A, FIG. 2B, FIG. 2C and FIG. 2D show an omega shaped synthetic ophthalmic graft patch of the invention used to cover an implantable device, such as a tube glaucoma shunt.

It will be appreciated that for simplicity and clarity of illustration, elements shown in the figures have not necessarily been drawn to scale. For example, the dimensions of some of the elements may be exaggerated relative to other elements for clarity. Further, where considered appropriate, reference numerals may be repeated among the figures to indicate corresponding or analogous elements.

### DETAILED DESCRIPTION OF THE PRESENT INVENTION

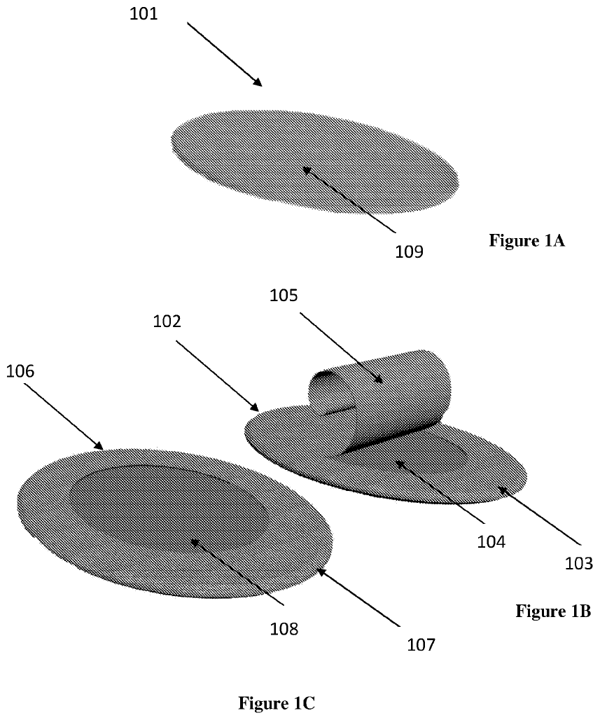

FIG. 1A. FIG. 1B and FIG. 1C shows the synthetic ophthalmic graft patch of the invention wherein its capacity in eyelid tarsus supplement procedures. FIG. 1A-1C shows a synthetic ophthalmic graft patch of the invention (101, 102 and 106) in the form of at least a part of the eyelid of a patient in need thereof, made of an electrospun porous polymeric structure (103, 107 and 109). The synthetic ophthalmic graft patch of the invention is shown in 102 and 106 wherein the anterior electro spun matrix (105) is peeled off (for visualization purposes only), showing the underlying rigid, synthetic, artificial tarsus (104 and 108).

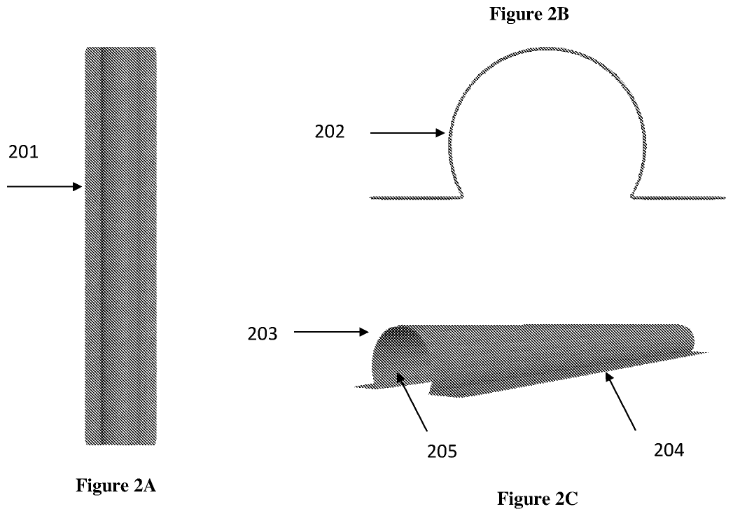

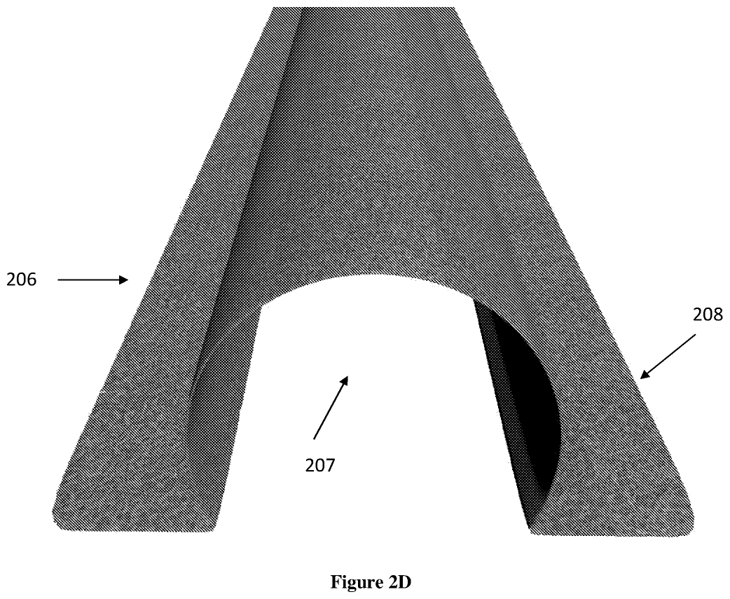

FIG. 2A, FIG. 2B, FIG. 2C and FIG. 2D show an omega shaped synthetic ophthalmic graft patch of the invention (201, 203 and in cross section 202 and 206) made an electrospun porous polymeric structure (205) which is formed to cover within its curved space (205, 207) an implantable device, such as a tube glaucoma shunt. Using such a synthetic ophthalmic graft patch of the invention, allows the sunt to be implemented in place without the need of a donor graft tissue, having higher degree of implantation success. The omega shaped synthetic ophthalmic graft patch of the invention is placed in position using also the optional flat bottom part (204 and 208).

While certain features of the invention have been illustrated and described herein, many modifications, substitutions, changes, and equivalents will now occur to those of ordinary skill in the art. It is, therefore, to be understood that the appended claims are intended to cover all such modifications and changes as fall within the true spirit of the invention.

1-12. (canceled)

13. A method of replacing and/or reconstructing and/or regenerating of ophthalmic tissue;
said method comprising implanting a synthetic ophthalmic graft patch having a porous polymeric structure with pores of less than 5 microns.

14. A method of replacing and/or reconstructing and/or regenerating of ophthalmic tissue;
said method comprising implanting a synthetic ophthalmic graft patch having a porous polymeric structure with pores of between 5 to 20 microns.

15. A method according to claim 13, wherein said synthetic ophthalmic graft patch is a biocompatible patch.

16. A method according to claim 13, wherein said synthetic ophthalmic graft patch is a biodegradable patch.

17. A method according to claim 13, wherein said synthetic ophthalmic graft patch has a thickness of between 50 to 250 microns.

18. A method according to claim 13, wherein said synthetic ophthalmic graft patch has a thickness of between 250 to 2500 microns.

19. A method according to claim 13, wherein said porous polymeric structure comprises at least one polymer.

20. A method according to claim 13, wherein said porous polymeric structure comprises nanofibers.

21. A method according to claim 13, wherein said porous polymeric structure comprises at least one porous electrospun polymer.

22. A method according to claim 13, wherein said porous polymeric structure comprises at least one polymer selected from poly(DTE carbonate) polycaprolactone (PCL), polylactic acid (PLA), poly-L-lactic acid (PLLA), Poly(DL-lactide-co-caprolactone, Poly(ethylene-co-vinyl acetate) vinyl acetate, Poly(methyl methacrylate), Poly(propylene carbonate), Poly(vinylidene fluoride), Polyacrylonitrile, Polycaprolactone, Polycarbomethylsilane, Polylactic acid, Polystyrene, Polyvinylpyrrolidone, poly vinyl alcohol (PVA), polyethylene oxide (PEO), polyurethane, polyvinyl chloride (PVC), hyaluronic acid (HA), chitosan, alginate, polyhydroxybuyrate and its copolymers, Nylon 11, Cellulose acetate, hydroxyappetite, poly(3-hydroxybutyric acid-co-3-hydroxyvaleric acid), poly(DL-lactide), polycaprolactone, and poly(L-lactide) or any combination thereof.

23. A method according to claim 13, wherein said synthetic ophthalmic graft patch further comprises at least one active agent.
